(19) United States
(12) Patent Application Publication
(10) Pub. No.: US 2022/0176143 A1
(43) Pub. Date: Jun. 9, 2022

(54) MODULE AND DEVICE FOR TREATING SYMPTOMS OF FIBROMYALGIA USING EMITTING ELECTROMAGNETIC WAVES

(71) Applicant: REMEDEE LABS, Saint Ismier (FR)

(72) Inventors: Laure MINIER, Meylan (FR); David CROUZIER, Meylan (FR); Michael FOERSTER, Corenc (FR)

(21) Appl. No.: 17/542,864

(22) Filed: Dec. 6, 2021

Related U.S. Application Data
(60) Provisional application No. 63/122,615, filed on Dec. 8, 2020.

(51) Int. Cl. A61N 2/00; A61N 2/02

(52) U.S. Cl. CPC A61N 2/008; A61N 2/02

(57) ABSTRACT

The present invention relates to a method for treating fibromyalgia and/or one of its symptoms in a human or animal subject, comprising the application of a portable device (10; 100; 1000) for transmitting electromagnetic waves to said human or animal subject, wherein said portable device is capable, when it is affixed at a surface (60), of transmitting waves having a power flux density of at least 0.5 mW/cm² of surface and a frequency value of between 3 and 120 gigahertz, the device being further capable of simultaneously exposing at least 2.5 cm² of the surface to the waves.

### TECHNICAL FIELD

The invention relates to the treatment of a chronic pain of low to moderate intensity, especially fibromyalgia.

### BACKGROUND

Fibromyalgia is a chronic disease causing pain, stiffness, and tenderness of muscles, tendons, and joints, in the absence of detectable inflammation. Other common symptoms include mental and/or physical fatigue and moderate to severe sleep disturbances. There is also an increased sensitivity to sensory stimuli, cognitive disorders and comorbidities of a psychiatric nature (such as mood disorders, anxiety and/or addictions).

The prevalence of fibromyalgia is difficult to assess because the definition of this disease is still unclear and subject to change. However, its prevalence is estimated at 2-4% in the general population, and increases drastically in clinical populations (i.e. 15.2% in rheumatology/internal medicine, 14.8% of patients with type 2 diabetes, and 12.9% of patients with irritable bowel syndrome). Fibromyalgia mainly affects women (between 80 and 90%), a rather young population (nearly 90% of patients are under 60 years old). In addition, there is a bidirectional relationship between fibromyalgia and psychiatric disorders, the development of one type of disorder increasing the likelihood of developing the other type.

Fibromyalgia is a complex and heterogeneous condition in which an abnormal pain process is associated with other clinical presentations.

To date, there is no specific treatment for fibromyalgia. Only symptomatic treatments and treatments to improve the patient's quality of life are available. The management recommended by the European League Against Rheumatism (EULAR, Macfarlane et al., 2017) is of a multidisciplinary type—combining drug and non-drug treatments—graduated, and personalized to improve quality of life.

Among drug treatments of fibromyalgia, analgesics (more than 30% of prescriptions), antidepressants (20% of prescriptions), antiepileptics and anxiolytic/hypnotic drugs are the most prescribed (HAS Report, 2010).

Analgesics include tramadol, with or without associated paracetamol, which is effective on pain and functional indices and quality of life. Tramadol has a dual synergistic action: a central opioid action due to binding to opioid receptors, and a central monoaminergic action due to the inhibition of norepinephrine and serotonin reuptake, a mechanism involved in the control of central nociceptive transmission. Antidepressants include serotonin and norepinephrine reuptake inhibitors. Antiepileptics, including pregabalin and gabapentin, act by modulating voltage-dependent calcium channels, thereby decreasing the release of substance P, calcitonin gene related peptide (CGRP), glutamate, and thus reducing sensitization of nociceptive voices.

To complement the effects of drug treatments, that do not always give convincing results and present non-negligible risks for patients (overdosage, potentiation of sedative drugs, risks of addictive behaviour, suicidal behaviour (e.g. pregabalin), numerous therapeutic alternatives are proposed to fibromyalgia patients and used by 98.1% of them. They include individualized graded physical exercise (that increase sleep quality, pain sensitivity threshold, exercise tolerance, well-being and self-confidence), massages (such as 20-90 minutes/massage, 10-20 sessions; they show effects on pain, anxiety and depression), thermal cures (such as 20 minutes bath/day, 5×/week for 3 weeks; they show effects on pain, quality of life and depression), whole body cryotherapy (such as 3 minutes at −140° C./session, 15 sessions in 3 weeks; it reduces nerve conduction and lowers tissue temperature), transcutaneous electrical neurostimulation: reduction of fatigue and hyperlagia, acupuncture (which reduces pain and stiffness, with better results of electric acupuncture compared to manual acupuncture on pain, stiffness, general well-being, sleep and fatigue), cognitive behavioural therapy (CBT), relaxation/sophrology/music therapy, and/or yoga/tai chi/qi-jong.

Most of these treatments involve the contribution of qualified medical staff (massage, CBT, acupuncture) and/or non-ambulatory equipment (spa treatment, cryotherapy), and are therefore expensive therapies, or require enough training to obtain benefits (relaxation/sophrology/yoga/tai chi).

Physical activity is EULAR's first recommendation for fibromyalgia patients, however, it often requires re-training for prior exercise and remains little practiced by patients who “at age 40 are as unfit as non-fibromyalgic 80-year-olds”.

An alternative to taking medication is to treat the pain by physical methods. The risk of side effects is then reduced, as well as that of drug dependence.

There is, in particular, a known form of treatment by transcutaneous electrical neurostimulation (or TENS for “Transcutaneous electrical nerve stimulation”) which consists in circulating an electric current in an area of the patient's body to stimulate nerves in order to reduce pain. However, the use of this technique requires a bulky device composed of electrodes to be placed on the patient's body and connected to an electric generator. In addition, it produces a tingling feeling which can be experienced as unpleasant by the patient. TENS devices are an interesting proposition because they offer patients autonomy of use as well as the portable aspect.

Lastly, it is a known fact that therapy by transmission of so-called “millimeter” waves (that is to say, whose frequency is less than 300 gigaHertz, i.e. between 30 and 300 gigaHertz) reduces pain (see the publication by Usichenko T I, Edinger H, Gizkho V V, Lehmann C, Wendt M, Feyerherd F: “Low-intensity electromagnetic millimeter waves for pain therapy. Evid Based Complement Alternat Med”). Indeed, it was shown that the exposure of an area of the human body to millimeter electromagnetic waves allowed the release of endogenous opioids (see the publication by Rojavin M A, Ziskin M C: “Electromagnetic millimeter waves increase the duration of anaesthesia caused by ketamine and chloral hydrate in mice Int J Radiat Biol”), generating in the brain the synthesis of enkephalin, a natural peptide involved in pain tolerance.

Therefore, this technique makes it possible to avoid the disadvantages of therapies with analgesics or by neurostimulation, and its modus operandi is understood. In addition, transmitting millimeter waves can also be used in the treatment of anxiety or sleep disorders.

Application WO2012/022538, based on this principle, discloses a device aimed at reducing pain in a patient by transmitting electromagnetic waves to the surface of the patient's body. The device is bulky and has an imposing horn shape which requires that the patient be brought to a specific location holding the device, or that the device be transported to the patient, who cannot therefore keep it on him and use it at any time and place. In addition, the patient must preferably be assisted by one or more persons, specially trained to operate the device. The significant volume of the device is largely due to the wave transmitting elements, such as the generator and the antenna(s), which must provide, in order for the treatment to be effective, frequency waves between 30 and 300 GHz, which must have a high power flux density.

The CEM Tech Company markets a less bulky device aimed at reducing pain by means of electromagnetic waves. Although this device is small, the level of power flux density of the transmitted waves is from 10⁻¹⁹ to 10⁻⁵ watts/cm². Now, it is known that the analgesic effect of millimeter waves is not observed for a power flux density of less than 0.5 mW/cm² (see the publication by Rojavin M A, Radzievsky A A, Cowan A, Ziskin M C: “Pain relief caused by millimeter waves in mice: results of cold water tail flick tests”.).

### SUMMARY OF THE INVENTION

Therefore, an object of the invention is to treat fibromyalgia effectively by means of electromagnetic waves, and to make the devices provided for this purpose accessible and easily usable by patients.

To this end, the invention provides a method for treating fibromyalgia and/or one of its symptoms in a human or animal subject, which comprises the application of a portable device for transmitting electromagnetic waves to said human or animal subject, wherein said portable device is capable, when it is affixed at a surface, of transmitting waves having a power flux density of at least 0.5 mW/cm² of surface and a frequency value of between 3 and 120 gigaHertz (GHz), the device being further capable of simultaneously exposing at least 2.5 cm² of the surface to the waves. By “application”, it is meant affixing said portable device to a skin surface of said human or animal subject.

The “human or animal subject”, also called “patient”, is a subject who is preferably a mammal. Preferably the subject or patient is human. Preferably, the subject or patient is afflicted by fibromyalgia.

Preferably, the method of the invention aims to treat at least one symptom of fibromyalgia in the human or animal subject. By “symptom of fibromyalgia”, it is meant at least one symptom chosen from (i) pain, (ii) stiffness, (iii) tenderness of muscles, tendons, and joints in the absence of inflammation, (iv) sleep impairments, and (v) fatigue/lack of rest. Preferably, the symptom of fibromyalgia is chosen from (i) pain, (ii) stiffness, (iv) sleep impairments, and (v) fatigue/lack of rest. Preferably, the method of the invention aims to increase the quality of life of a human or animal subject afflicted by fibromyalgia. By “quality of life”, it is meant an improvement of at least one of the symptoms of fibromyalgia, optionally combined with the lack or decrease of anxiety and/or depression and/or the improvement of physical functioning.

Thus, the device is portable, that is to say, capable and intended to be worn by a patient on a regular basis, or even continuously, without significant effort and without logistical or practical disadvantages. In addition, the power flux density of at least 0.5 milliwatts per square centimeter of surface allows the treatment to be effective and to reduce pain. Finally, the frequency is a frequency band particularly effective for the treatment by millimeter waves. Moreover, the study by Radzievsky A A, Gordiienko O V, Alekseev S, Szabo I, Cowan A, Ziskin M C: “Electromagnetic millimeter waves for pain therapy Evid Based Complement Alternat Med” tends to show that the optimal effect of a treatment by millimeter waves is obtained with a frequency around 61.25 GHz and a power flux density of approximately 13 mW/cm².

The device used in the invention is relevant, because it relies on one's own resource. Thus, there is no risk of overdose, nor any other side effects. Preferably it is a wearable device such as a wristband. Said therapeutic wristband is a “at home” treatment which can be used at convenience. The patients are thus completely autonomous and responsible for their own treatment in terms of time and frequency, an aspect that is poorly addressed amongst the current solutions offered to fibromyalgia patients.

Concerning the aptitude to expose at least 2.5 cm² of surface, the device may, for example, present a module comprising several antennas transmitting waves simultaneously, the area covered by all the antennas, and therefore by the module, representing at least 2.5 cm²exposed to waves in a homogeneous way. This makes it possible to obtain a surface continuously exposed to waves in a manner sufficient to induce the expected biological response.

Alternatively, the module may be capable of discontinuously exposing 2.5 cm² to waves, that is to say, several surface portions distributed over several different locations which, all together, represent 2.5 cm² of simultaneously irradiated surface.

It should be noted that the frequency transmitted by the different antennas is not necessarily the same. The different antennas may transmit different frequencies, being supplied by different application specific integrated circuits (ASICs). Nevertheless, the frequencies remain within the band of interest.

Advantageously, the waves have a power flux density of between 5 and 35 mW/cm², preferably of between 5 and 15 mW/cm².

Thus, it is a particularly efficient power band. On the other hand, some standards do not allow a power density above a certain threshold, so that the device may be calibrated so as not to exceed this limit, if necessary.

Preferably, the surface being human or animal skin, the device comprises a unit for detecting human or animal skin, the device being capable of signaling the presence or absence of the skin to be exposed to waves, and preferably capable of determining a distance between the skin and the device.

“Exposing to the waves” also means “irradiating by the waves”.

Thus, the device transmits waves directly to the subject's skin only if the skin is detected. If the skin is not detected, or if the distance between the device and the skin is too great, no transmission takes place. This prevents sending waves in any direction and allows saving energy. It is also possible to adapt the power or other parameters of the waves transmitted based on the estimated distance between the device and the skin.

Advantageously, the device is able to be worn at least in one of the following sites:
around a wrist;
on a leg;
on an ankle;
in the back;
on an ear;
in the palm of a hand; or
more generally any site presenting a strongly innervated area.

Thus, it is affixed at one of these sites, for example, around the wrist, like a watch, so as to be worn without particular inconvenience for the patient. Concerning heavily innervated areas, the study “Radzievsky A A, Rojavin M A, Cowan A, Alekseev S I, Ziskin M C. Hypoalgesic effect of millimeter waves in mice: Dependence on the site of exposure. Life sciences. 2000; 66 (21): 2101-11” demonstrated the beneficial therapeutic effect of sending millimeter waves in such areas. The device of the invention further presents the advantage of being wearable by the subject. This avoids going into a therapeutic center.

Preferably, the device comprises a rechargeable battery.

Thus, it works wirelessly. Alternatively, it may operate by wire, in order to deliver higher powers or over longer durations.

Preferably, the device comprises a heat sink comprising at least one of the following elements:
a flexible material;
a phase change material;
a thermal buffer;
graphite; and
an elastomeric material.

Thus, the heat sink makes it possible to minimize heating of the device in order to maintain it at a temperature below 43° C. If it is flexible, it allows it to adapt to the shape of the device and its maneuverability.

Advantageously, the device comprises a unit for determining at least one data from the surface, for example, an impedance data.

Thus, this is carried out after transmitting the electromagnetic waves, by measuring the output power which is based on the adaptation of relative impedance to the skin. The device thereby obtains automatically one or more characteristics of the patient's skin.

Preferably, the device comprises a processing unit making it possible to deduce any variation of the determined data.

Thus, the data obtained are autonomously processed by the device which itself adapts the parameters of the waves, without any intervention by the patient who only has to activate or program the wave transmission without worrying about settings and adapting the waves to his own body.

The device used in the invention may also comprise a module for transmitting electromagnetic waves, which has a total volume of less than 4 cm³, preferably less than 3 cm³, and is suitable, when it is arranged at a surface, to transmit electromagnetic waves having a power flux density of at least 0.5 mW/cm² of surface.

Thus, this module is particularly miniaturized, which allows it to be integrated into portable devices or to transport and use it easily. The minimum power flux density of 0.5 mW/cm² is that from which millimeter wave treatment is effective for treating pain, but this module may be intended for other uses.

Preferably, the administration of electromagnetic waves to the subject according to the invention is made during a period of 15 minutes to 40 minutes, preferably from 30 to 40 minutes. Preferably, said administration is performed one, twice or three times a day. Preferably, said administration is performed after wake-up, and/or within one hour before bed time, and/or before or after a muscular effort, and/or before or after a static period.

The invention also provides a method for treating fibromyalgia and/or one of its symptoms in a human or animal subject, which comprises a step of transmitting electromagnetic waves towards the subject's skin, thanks to a transmitter worn by said subject, said waves having a power flux density of at least 0.5 mW/cm² of skin and a frequency between 3 and 120 GHz, preferably between 50 and 100 GHz, preferably between 60 and 95 GHz. More preferably, said waves have a frequency of between 61 and 61.5 GHz.

Advantageously, the method comprises the following steps:
a unit detects human or animal skin, and
when the unit detects that the skin is located at three millimeters or less from the transmitter, the transmitter transmits the waves.

Another predetermined distance may be considered. The distance of 3 mm corresponds to a distance close enough to allow the skin to absorb almost all of the power of the electromagnetic radiation, even if the module is not in contact with the skin, for example, when moving the device or if the latter is not affixed by being pressed against the patient's body.

Preferably, the method includes the following steps:
determining at least one impedance data of the skin; and
depending on the or each data, adapting at least one transmission parameter.

Preferably, the wave transmission takes place at least one predetermined acupuncture point of the subject.

Wearing the device on the wrist, in particular on the acupuncture point Pericardium 6, the wrist being an area particularly rich in nerve endings, makes it possible to increase the effectiveness of the millimeter wave treatment.

Advantageously, the transmission is controlled at the transmitter or by means of a device able to communicate with the transmitter through a telecommunication network.

The method of the invention may be performed using a computer program comprising code instructions capable of controlling the implementation of the steps of the method described above when it is executed on a computer.

This computer program may be included in computer software but also in an application for a mobile terminal such as a smartphone or a “smartwatch”, otherwise called “connected watch”.

The invention also provides a method of accessing the program according to the invention for downloading onto a communication network.

Lastly, the invention provides a device comprising telecommunication means and the program according to the invention.

Thus, this device may be, for example, a computer or a smartphone.

According to another aspect, the method of the invention is performed using a wave transmission module, which has a total volume of less than 4 cm³, preferably less than 3 cm³, and is capable, when it is placed on a surface, to transmit electromagnetic waves having a power surface density of at least 0.5 mW/cm² of surface.

Thus, this module of small size may be integrated into an easily handled device, for example, portable, such as a smartphone or a smartwatch, or be integrated in large numbers into a more complex device generating high radiation without taking up a large space within the device. In addition, starting from 0.5 mW/cm², it is known that an effect in the treatment of pain is obtained (see the publication by Rojavin M A, Radzievsky A A, Cowan A, Ziskin M C: “Pain relief caused by millimeter waves in mice: results of cold water tail flick tests”), so that only one of these modules, of small size, can allow the therapeutic treatment of a patient or serve other applications such as stress reduction, generation of a feeling of well-being or the resolution of sleep disorders, without taking up space, and with reduced cost.

Advantageously, the waves have a power surface density value of between 5 and 35 mW/cm², preferably of between 5 and 15 mW/cm².

Thus, the waves transmitted comply with certain standards limiting their power towards human skin, but the power remains sufficient for an effect to be obtained, for example, a reduction in pain or a feeling of well-being.

Preferably, the waves have a frequency value between 3 and 120 GHz, preferably between 50 and 100 GHz, preferably between 60 and 95 GHz. More preferably, said waves have a frequency of between 61 and 61.5 GHz.

This is a particularly effective frequency band for the treatment of pain using millimeter waves.

Advantageously, it includes a rechargeable battery.

Therefore, it operates wirelessly. Alternatively, it may operate by wire, in order to deliver higher powers or over longer periods of time.

Preferably, the module is able to simultaneously expose at least 2.5 cm² of the surface to the waves. Thus, the module is capable of continuously exposing 2.5 cm² of a surface, in particular, skin, to the waves. Alternatively, the module may be capable of discontinuously exposing 2.5 cm² to waves, that is to say, several surface portions distributed over several different locations which, all together, represent 2.5 cm² of simultaneously irradiated surface.

For example, the module has several antennas transmitting waves simultaneously, the skin area of a patient covered by all of the antennas, and therefore by the module, representing at least 2.5 continuous cm², irradiated in a homogeneous manner. This provides a continuous irradiated surface sufficient to induce the expected biological response. It should be noted that the frequency transmitted by the different antennas is not necessarily the same. The different antennas can transmit different frequencies, being supplied by different ASICs. Nevertheless, the frequencies remain within the band of interest.

Advantageously, the module comprises a heat sink comprising at least one of the following elements:
a flexible material;
a phase-change material;
a thermal buffer;
graphite; and
an elastomeric material.

Thus, the heat sink makes it possible to minimize the heating of the module, in particular if it is integrated into a device applied to the skin of a patient. This, again, makes it possible to comply with certain standards but also, more simply, to avoid excessive heating of the module or of the device in which it could be integrated.

Preferably, the surface being human or animal skin, the module comprises a skin detection unit capable of signaling the presence or absence of the skin to be exposed to the waves, and, preferably, capable of determining a distance separating the skin and the module.

Thus, the module transmits waves directly towards the subject's skin only if the skin is detected. If the skin is not detected, or if the distance is too great between the module and the skin, no transmission takes place. This way, sending waves in any direction is avoided and energy is saved. The power or other parameters of the waves transmitted may also be adapted according to the estimated distance between the module and the skin.

The invention further provides a portable device for transmitting electromagnetic waves, comprising a module described above.

Thus, the device can be easily worn by a human or animal patient and send waves in a predetermined manner or on command, for a therapeutic purpose, in order to generate a feeling of well-being or for any other purpose. The device is all the easier to be worn since the transmission module is small.

Preferably, the device is able to be worn at least in one of the following places:
around a wrist;
on a leg;
on an ankle;
on the back;
on an ear;
in the palm of a hand;
or more generally, any place presenting a highly innervated zone.

Thus, it is affixed at one of these sites, for example, in the manner of a watch around the wrist, so as to be worn by the patient without particular inconvenience.

An electromagnetic wave transmission method is also provided according to the invention, in which a module described above, worn by a human or animal subject, transmits electromagnetic waves having a power flux density of at least 0.5 mW/cm² of skin towards the skin of the subject.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a block diagram of an embodiment of the invention;

FIGS. 2 and 3, 5 and 6 are illustrations of a portable device according to a first embodiment of the invention;

FIG. 4 illustrates a first embodiment of such a device;

FIGS. 7 to 15 are diagrams of the components of a wave transmission module according to a first embodiment;

FIGS. 16 and 17 are illustrations of such a module respectively without and with heat sink;

FIG. 18 is an illustration of a radiation of the module of FIGS. 14 and 15;

FIGS. 19 and 20 are illustrations of use according to the second and third embodiments of the invention;

FIGS. 21 to 29 illustrate components of a module according to other embodiments of the invention;

FIG. 30 illustrates a flow chart of an example process for treating fibromyalgia symptoms, according to certain aspects of the disclosure;

FIGS. 31 and 32 illustrate results of a fibromyalgia treatment program; and

FIG. 33 illustrates an electronic system with which one or more implementations of the subject technology may be implemented.

### DETAILED DESCRIPTION

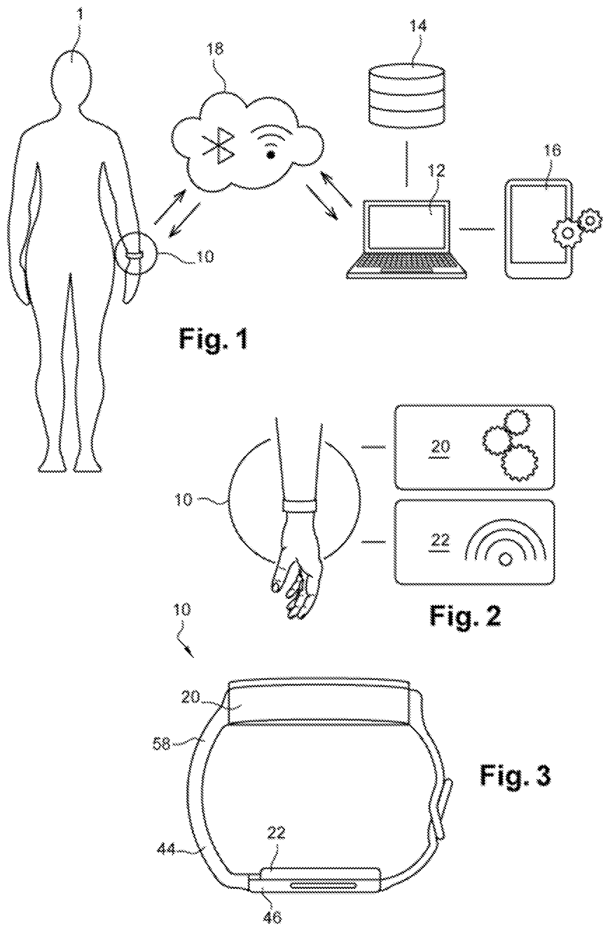

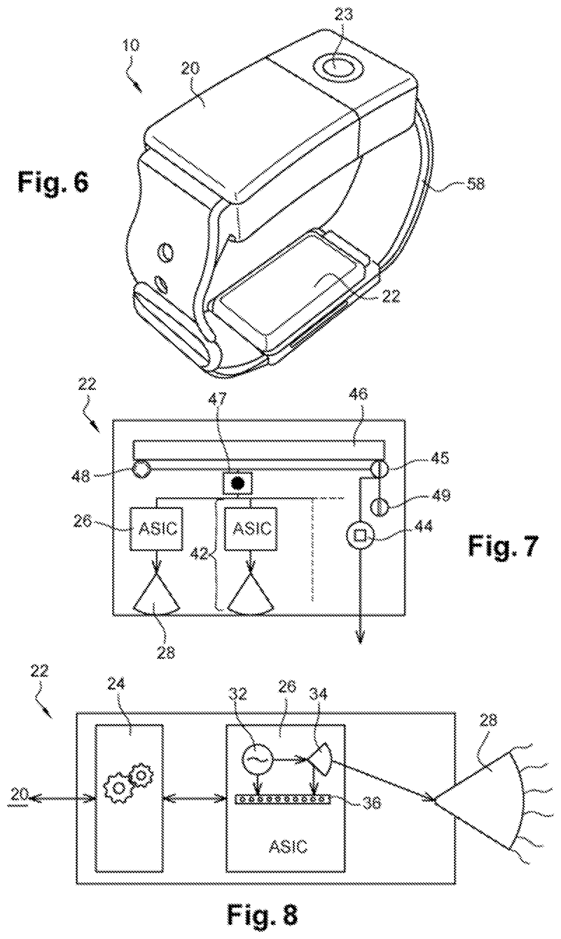

FIG. 1 illustrates the general framework of the invention. Patient 1 has chronic pain. He wears a device 10, according to a first embodiment and a first implementation of the invention, which treats the pain by transmitting electromagnetic millimeter waves to the skin of patient 1's wrist. In this case, this device 10 is in the general form of a wristwatch, and is affixed around the wrist in the same way as a watch. The device 10 comprises a control module 20 and a wave transmission module 22 illustrated schematically in FIG. 2 and in more detail in FIGS. 3, 5 and 6. The device 10 being in the general form of a watch, it may be a watch in which the modules 20 and 22 are integrated. Conversely, the functions of a watch may be integrated into device 10.

The control module 20 controls the transmission module 22. The control module 20 is activated by the patient, but it may also be programmed by the patient or another user, on the device 10 directly with the button 23 or via a terminal such as the computer 12. The button 23 is provided with light-emitting diodes which can be activated to indicate an event to the patient, for example a lack of battery or the operation of a particular program in progress. The control module 20 is present in the upper part of the device 10 while the millimeter wave transmission module 22 is located in the lower part and therefore intended to be in contact with the skin of the lower part of the wrist.

The wave transmission module 22, integrated into the device 10, will now be described in detail. It is a transmission module according to a first embodiment. This type of module, as well as its other embodiments, may be integrated into any type of device aimed at transmitting waves, and not only into the device 10 in the form of a wristwatch. Its applications are not limited to the treatment of pain.

This transmission module 22, schematically illustrated in FIG. 7, presents several circuit-antenna pairs 42, a heat sink 46, a skin sensor 44, a power input 45, a digital control unit 47, a reference clock 48, and a temperature sensor 49.

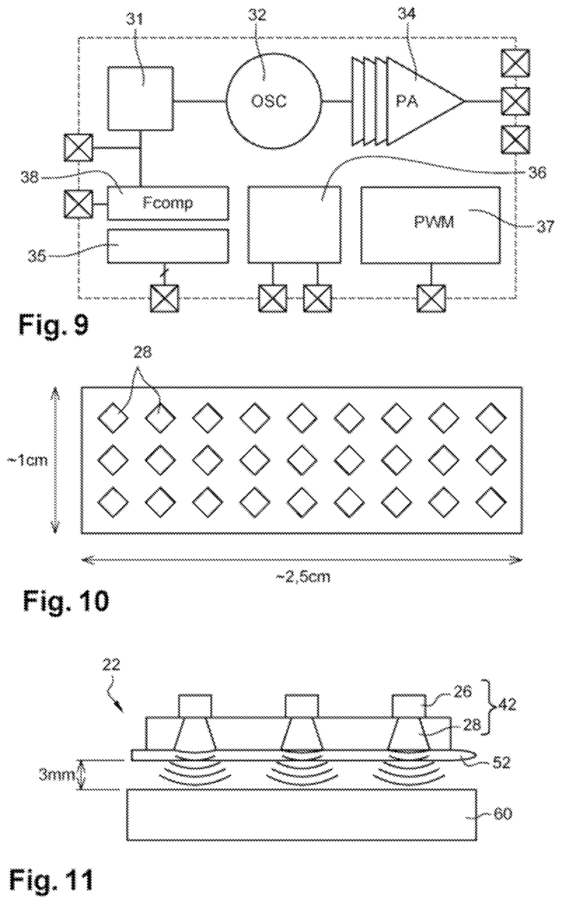

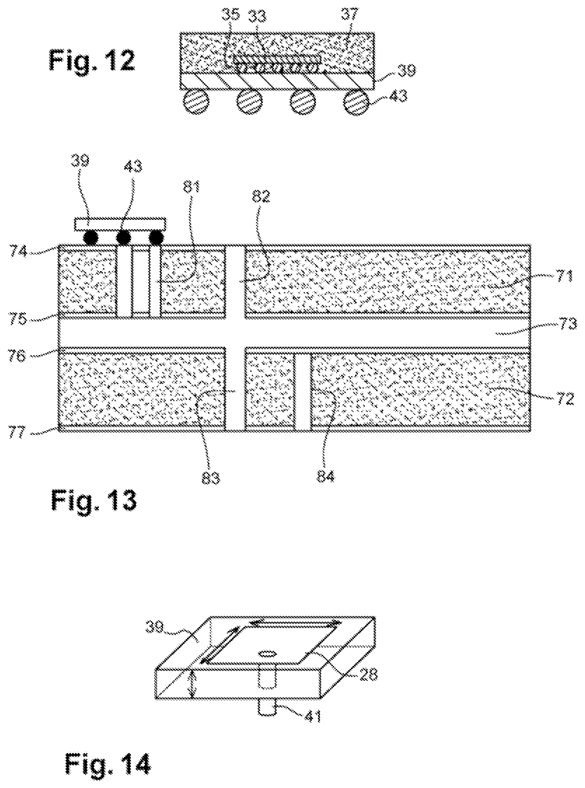

Each circuit-antenna pair 42, one of which being diagrammatically illustrated in FIG. 8, presents a control interface 24 in connection with the control module 20, an ASIC (“application-specific integrated circuit”) 26 and an antenna 28. The interface 24 may be located within the control module 20. The ASIC 26, as illustrated in FIG. 8, comprises an oscillator 32, a power amplifier 34 and a digital part 36 for setting and controlling the component. Illustrated in more detail in FIG. 9, it also includes a frequency divider 31, a communication bus 35, a “pulse-width modulation” (PWM) control unit 37 and a frequency comparator 38. The oscillator 32 allows the ASIC operating frequency to be generated. The amplifier amplifies this signal so that the desired power is available at the component output. This power is adjustable between 0 and 20 mW. It may conceivably go up to 60 mW without any difficulty. The frequency comparator and the divider make it possible to check the operating frequency. The power management circuit makes it possible to supply all of the component's functions correctly. Finally, the “PWM” control unit makes it possible to transmit the HF output signal continuously or discontinuously. The framework of the ASIC is shown in FIG. 12. This ASIC 26 is manufactured using complementary metal-oxide semiconductor (CMOS) technology, which is known to the person skilled in the art and will not therefore be described in detail. More specifically, the transistors are of the “CMOS 65 nanometer” type. Alternatively, they could have been developed in silicon-germanium (SiGe) or even in gallium arsenide (GaAs). On the other hand, technologies of the “Gunn diode” type do not allow to achieve the desired minimum size and cost. Thus, the ASIC 26 includes a silicon integrated circuit 33 housed in a ball grid array (BGA)-type housing 37, a type of housing well known to the person skilled in the art, tailor-made for the ASIC 26, the housing also comprising balls (called “bumps”) 35. As illustrated in FIG. 13, the circuit 33 is welded on two layers 71 and 72 of “HF” substrate 39 made of PTFE (Polytetrafluoroethylene) RO3003, for example, manufactured by Rogers, with an arrangement known as a “flip chip”, which makes it possible to minimize the losses of electromagnetic radiation at high frequency. An alternative to RO3003 could be MT77 (for example, from Isola) impregnating woven glass fibers, or even RF301 (from Taconic), or any other material offering the same technical advantages as those mentioned. The two layers 71 and 72 are separated by a layer 73 of RO4450F as well as by copper layers 74, 75, 76 and 77. In addition, vias 81, 82, 83 and 84 make the connections between the different layers of the substrate. Understandably, the types of layers and their number could be different.

The frequency oscillator 32 is placed in a cavity (not shown) within the housing 37 which allows not to disturb the generated frequency. The size of this BGA housing 37 is, in this case, 2.2×2.2×0.9 millimeters. The connection to the antennas 28 is made by means of “balls” 43. This set of components makes it possible to minimize the losses of electromagnetic waves. It is the antenna 28 which transmits electromagnetic waves to the skin of the patient 1. Needless to say, the arrangement of the ASICs, control interface and antennas within the transmission module may be different.

The terminal connection 41 between an ASIC 26 and its antenna 28 is visible in FIG. 14. Thus, a coaxial connection 41 ensures the transmission of the wave between the power amplifier 34 and the antenna 28. Antenna means generally any form of radiating element, provided that it is flat. This type of radiating element can be called a “patch”.

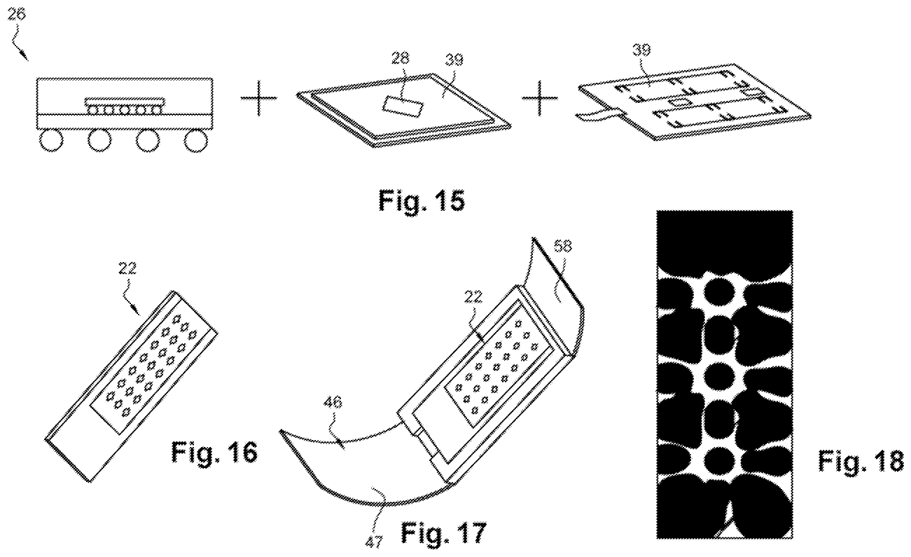

As shown in FIG. 15, the ASIC 26 and the antenna 28 are arranged on either side of the substrate 39.

The set of antennas 28 forms an array of antennas, illustrated in FIG. 10. Rectangular-shaped here, this array of antennas, intended to be placed against the skin of patient 1 or at a short distance from it, is approximately 2.5 centimeters long by about 1 centimeter wide. It is provided, in this case, with 27 radiating elements 28 operating in near field, on the basis of three rows of nine antennas, aligned vertically and horizontally with respect to each other. These quantities and these arrangements are not limiting and others may be considered. The other elements of FIG. 7 and FIG. 9, in particular the temperature sensor 49, the skin sensor 44, the clock 48 and the power module 45 are arranged around this array of antennas, also called active area, as illustrated in FIG. 22 in a slightly different embodiment described below. The assembly formed by these elements and the active area located inside measures 37×20 mm and forms the transmission module 22, which may be integrated into a device such as a bracelet.

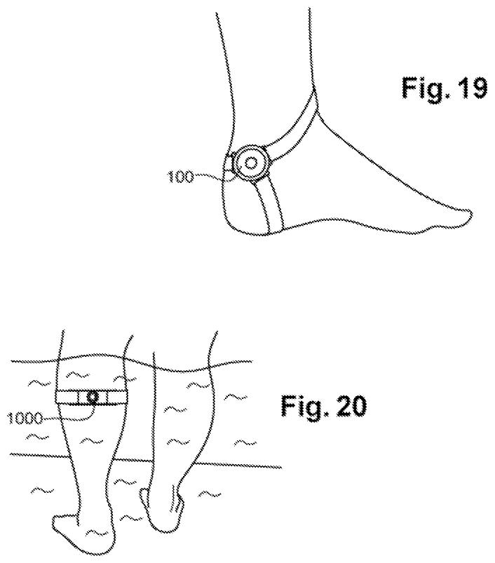

This arrangement allows the active area to transmit waves homogeneously over 2.5 square centimeters of skin. “Homogeneous” means that the intensity of the waves arriving on the skin must not present a deviation greater than about 30% between its maximum value at one point and its minimum value at another. FIG. 18 shows the radiation emitted by the device on the patient's skin in a normal operating mode. The black forms correspond to radiation between 5 and 15 mW/cm², the white forms to radiation below 5 mW/cm². It is observed that 75% of the surface is irradiated by waves of density between 5 and 15 mW/cm². In general, the power density can be greater than 35 mW/cm², but the device is designed so that the power range used is of the order of 5 to 35 mW/cm² in normal operation, in particular for 30 minutes of continuous wave transmission. This operating mode is indeed the most usual, as will be described below.

FIG. 11 illustrates an application of the module 22 for transmitting waves towards the skin 60 of patient 1. A distance of 3 millimeters separates the module from the skin of the patient in this case. Although the objective is to affix the device to the skin, it may happen that a slight space is created between the skin and the device. Furthermore, for more comfort and for reasons of biocompatibility, a silicone layer 52 separates the antennas from the skin, so that the skin does not have to directly support the antennas. Alternatively, it may be another material transparent to millimeter waves, such as polycarbonate. This layer 52 of silicone may measure from 1 to 2 millimeters, the design of the antennas allowing the layer 52 to have only little or no interference with the waves emitted.

Overall, this wave transmission module 22, which can be called millimeter module (the waves being said to be “millimeter” in view of their frequency) or millimeter card, measures 37 millimeters in length, 20 millimeters in width and is 3 millimeters thick in this embodiment. Therefore, the volume of the millimeter module is 2.96 cm³. As shown in FIG. 16, it is therefore less than four, and even less than three cubic centimeters, which makes it possible to insert it into light low-volume devices, such as the device 10 in the form of a wristwatch. Having this volume and the described arrangement presenting 27 antennas, the ASICs 26 developed, coupled to the antennas 28, allow the millimeter module to transmit waves of frequency between 3 and 300 GHz, preferably between 30 and 120 GHz. The preferred frequency is 61.25 GHz+/−250 MHz. In all cases, the power flux density is at least 0.5 mW/cm², and the waves are transmitted simultaneously on a skin surface of 2.5 cm². However, a millimeter wave treatment is effective at a power density starting from 0.5 mW/cm², preferably on a surface of at least 1 cm². Therefore, the disclosed module makes it possible to carry out the treatment because it is easily integrated into any device due to its small volume.

It is understood that the ASICs, the antennas, as well as the whole of the millimeter module 22, may have different volumes, numbers and arrangements.

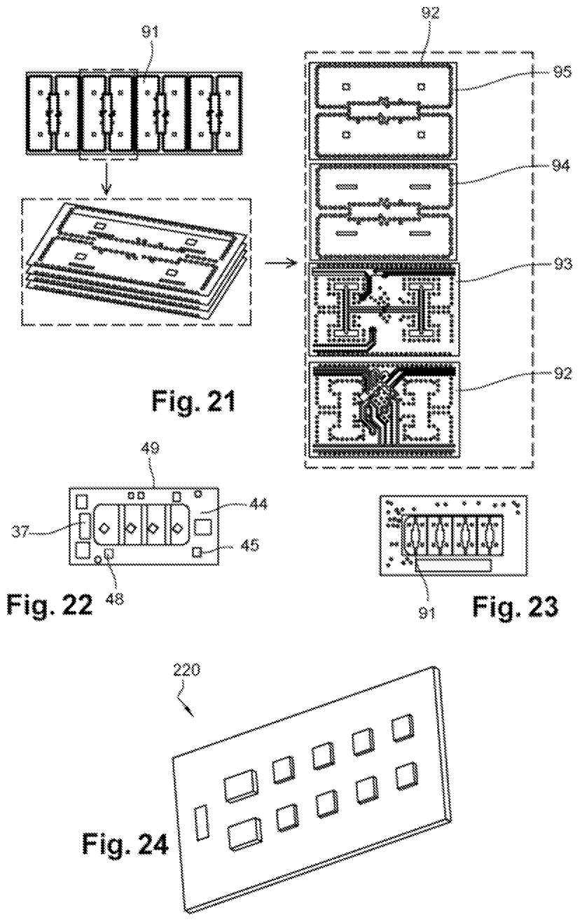

Thus, in a second embodiment, illustrated in FIGS. 21 to 23, the module's performances are identical. The difference is that an ASIC is coupled to four antennas on a surface of 10×6.25 mm. Therefore, this ASIC/antenna pair covers a skin surface of 0.625 cm², on a PCB substrate 1 mm thick. Repeated four times side by side in the millimeter module illustrated in FIGS. 21 to 23, the four ASICs are each placed in a different “BGA” housing, whose size is 2.2 mm×2.2 mm×1 mm. The module, which then includes two rows of eight antennas and 4 ASICs (4 housings), thus makes it possible to continuously irradiate 2.5 cm² of skin surface.

An antenna array 91 according to this embodiment is illustrated in FIG. 21. Array 91, said to be a “resonant cavity” array, comprises four layers. The layer 92 allows the routing of digital and power signals. The second layer 93 represents the access lines to the antennas. The third layer 94 represents the coupling lines. Lastly, the fourth layer 95 is that from which the waves will be transmitted. This antenna array is also implemented in the previous embodiment, the only difference being the number of antennas and ASICs, and, therefore, the arrangement of these elements.

Alternatively, by placing the ASIC/four antenna pairs separately at different locations on the patient's skin, this 2.5 cm² surface is irradiated, but in several distinct areas. Likewise, each of these pairs may be used independently in order to ensure greater comfort or to be integrated into applications which require a smaller surface, or a lower power.

The skin sensor 44 of the embodiments described uses a capacitive type measurement making it possible to determine that the patient's skin is positioned near the millimeter module 22. Its structure is known to the person skilled in the art and is not limited to a capacitive measurement, any miniaturizable skin sensor being admissible. Connected to the control interface 24 and/or to the control module 20, the skin sensor 44 determines the presence or absence of human or animal skin. It is also able to determine the distance between the skin and the millimeter module. At 3 millimeters or less, wave transmission is allowed. Otherwise, the control module 20 can prevent the wave transmission. The objective here is to prevent inefficient wave transmission in order, on the one hand, to control the direction of the waves transmitted, and, on the other hand, to save energy. In the first embodiment, the skin sensor 44 is located outside the module, on a side of the device 10.

The millimeter module 22 may further comprise a rechargeable battery. Preferably, the device assembly comprising the module 22, such as the device 10, has a battery supplying both the control module 20 and the wave transmission module 22. This battery can be recharged conventionally from the mains or any other way. It is, naturally, interesting that its autonomy is several hours, even several days, so that the patient's portable device aimed at treating his pain is more convenient to use.

Some of the module components may needless to say, be placed outside thereof to better interact with the device comprising the module, such as the battery.

Apart from the control module 20, the millimeter module 22 and the skin sensor 44, the device 10 includes other components which will be described now.

The band 58 of FIG. 3 is flexible and aims to adapt to the shape and size of the wrist, as would a conventional watch strap.

The device 10 also includes a dissipator 46, shown in FIG. 5, which may be considered as part of the millimeter module 22. In the present case, it is located outside this module, and comprises a flexible band 47 and a thermal buffer 48, the two components being inserted within the strap of the device 10. The band 47 is associated with graphite and rubber. The rubber allows the band to be flexible and therefore adaptable to the strap. The graphite is light and has good thermal conductivity. The band 47 may be made of another elastomeric material than rubber. It may also be made of a completely different material, which needs to be flexible in order to adapt to the shape of the device. The buffer 48 includes a phase-change material. Thus, during heat release due to the operation of the device, the phase-change material absorbs part of the calories generated and allows to maintain the overall temperature. The dissipator is arranged with the device in order to maintain the temperature of the surrounding area of the body below 43° C. for a continuous operation of the device of approximately 30 minutes. This temperature of 43° C. corresponds to maximum temperature standards set by certain authorities, and that is why the device is designed to comply with these standards. It could thus be designed differently if the maximum authorized temperature were higher. The temperature is monitored by the temperature sensor of the millimeter module 22.

The device 10 further includes a unit (not shown) for determining the impedance of the skin. This unit may be part of the millimeter module 22.

The frequency of the waves transmitted by the device 10 via the module 22 may be between 3 and 300 GHz for an effective treatment. However, the frequency of the device disclosed preferably varies between 30 and 120 GHz, with a preferred frequency around 60 GHz, in particular around 61.25 GHz.

Each component's dielectric properties, such as its permittivity, conductivity and loss tangent, had to be taken into account for the design of the module 22 and the device 10. Simulations and tests outside the nominal operating range of the 65 nm CMOS type ASIC transistors were carried out, and do not call into question the lifetime of the components with regard to the implementation of the millimeter wave treatment which will be disclosed below.

The implementation of pain treatment in the patient will now be disclosed.

This treatment aims to transmit waves towards an area of the patient's skin. The transmission generally lasts 30 minutes, at the rate of one transmission to two per day. The frequency, preferably between 30 and 120 GHz, is predetermined. It may possibly vary during a transmission, as does the power flux density which generally varies between 5 and 35 mW/cm², but can be lower or higher than this range. Needless to say, any other type of treatment is possible, in particular with longer and/or more frequent transmissions.

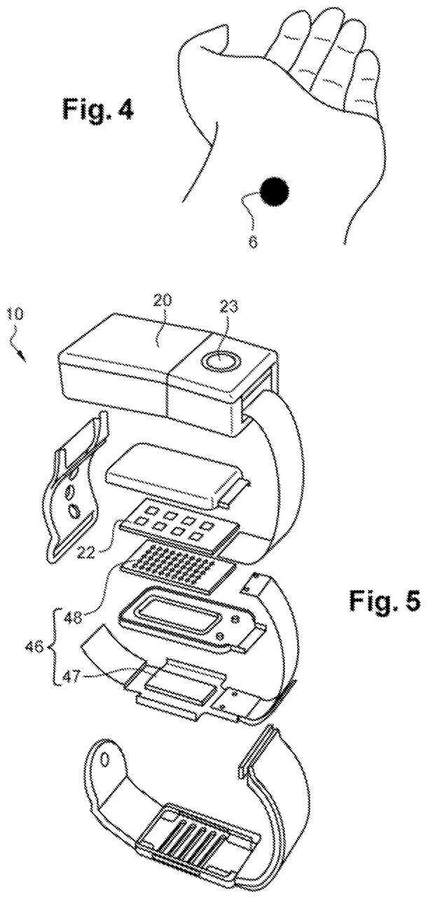

In a first embodiment, the waves are transmitted by the module 22, integrated into the device 10 in the form of a wristwatch, towards the wrist, a highly innervated area, and may be placed on the acupuncture point Pericardium 6 referenced in FIG. 4, which is a known acupuncture point. It has indeed been shown that the transmission of waves to acupuncture points was particularly effective in the treatment of pain. In addition, very good results are also achieved for particularly innervated areas. Indeed, the stimulation of the nerve endings located under the skin induces a set of physiological actions called “systemic response”, actions which in turn induce the synthesis of endogenous opioids (including the enkephalin) themselves responsible for decrease in pain. Thus, the more the wave transmission takes place in an area with a high density of nerve endings, the more the treatment is likely to be effective. Point Pericardium 6 is an acupuncture point at the same time located in an area rich in nerve endings. Therefore, a device transmitting waves towards this area is of utmost interest.

Furthermore, other potential benefits, described in the literature associated with this increase in the synthesis of opioids, are known, such as a decrease in heart rate and stress, improved sleep, or even a euphoric effect. Therefore, such benefits can be drawn from the device 10.

The frequency, the duration, and the power of the waves can be parameterized by means of the module 20 of the device 10. As illustrated in FIG. 1, it can be programmed in advance by means of a terminal, for example a computer 12, which can communicate with it by any telecommunication network, such as a Bluetooth or Wi-Fi type link 18. The computer 12 includes a database 14 on which is recorded a program 16 implementing the process or processes having a link with the invention, as well as various data allowing the implementation of the invention, in particular data entered by the patient 1 and data obtained by the device 10.

In addition, by determining the impedance of the skin using the impedance detection unit, the latter transfers to the control module 20 a characteristic data of the patient's skin. Parameters of the waves transmitted by the module 22 can then be modified automatically via the control unit 20, thanks to the program 16, or manually by the patient or another user. Thus, the device 10 adapts to the patient's skin. In other words, the electromagnetic field created is controlled by the characteristics of the skin. It can also be modified based on the distance measured between the skin and the device, via the skin detector 44. The device may include other units determining and processing other data obtained directly from the patient, which can serve to adapt the parameters of the transmitted waves such as power, frequency and duration of transmission.

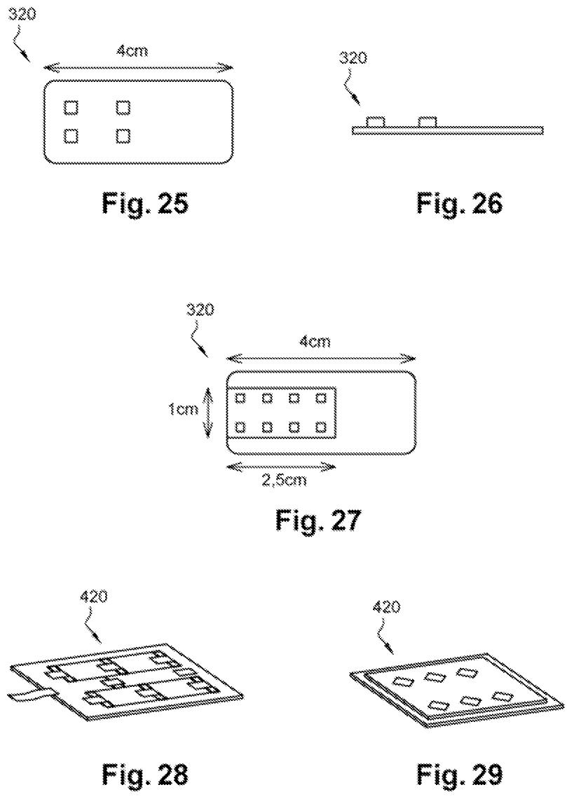

Other embodiments of the transmission module are illustrated in FIGS. 24 to 29. They differ from the previous embodiments by their number of ASICs and antennas. Thus, the module in FIG. 24 comprises 8 ASICs. In addition, one or more radiating elements may correspond to an ASIC. Thus, the module 320 comprises 4 ASICs for 8 radiating elements, at the rate of 2 radiating elements for 1 ASIC. Finally, module 420 comprises 6 ASICs and 6 radiating elements.

Furthermore, the transmission module may also be integrated into another device, for example intended to be worn by the patient in another part of the body. Thus, FIG. 19 illustrates a device 100 according to a second embodiment comprising the control and transmission modules placed on the ankle, while FIG. 20 illustrates such a device 1000 according to a third embodiment placed on the calf. Therefore, in these second and third embodiments, the waves are transmitted to other areas of the patient's body by means of devices which differ from the device 10 essentially to adapt to the targeted area of skin. In all cases, the miniaturization of the modules allows the device to be light and not bulky, so that it is easy to wear and not excessively burdensome.

Modifications are possible within this transmission module. For example, the structure of the antenna array may be different and present a “micro ribbon” type supply line or a coaxial probe. The antennas may be long slot antennas.

The control module may also be integrated into the electromagnetic transmission module.

Therefore, several embodiments and implementation modes were presented, which all allow the transmission of electromagnetic waves having a power surface density of at least 0.5 mW/cm² of surface, a frequency value between 3 and 120 GHz, and simultaneously on a surface of at least 2.5 cm², whether continuous or spread over several separate parts of the surface.

Aside from any pain treatment, the wave transmission module, possibly in conjunction with the control module, may be interesting for transmitting waves for other purposes, for example, to improve sleep, since it is particularly miniaturized, and therefore light. Consequently, it can be integrated into any device when it is necessary to send millimeter waves to a surface or in any direction.

Furthermore, the transmission module, or the control module, and/or the device integrating these modules, may be controlled remotely, from a terminal such as a computer, but also from a mobile terminal. For example, a mobile application comprising a pain treatment program may be saved on the mobile terminal, so that the patient programs his treatment himself, for example the power, the frequency, the duration and the time of wave transmission, or his doctor or any medical assistant programs these parameters remotely. In this case, the terminal comprises software presenting one or more interfaces allowing the user of the terminal to configure the device. The program allowing the implementation of the invention may be downloaded via a telecommunication network.

It may be added that the transmission module, as well as the device comprising it, may also be used in order to reduce the patient's stress or even bringing a feeling of well-being.

As a corollary, one can envisage the use of the transmission of electromagnetic waves within the framework of a program of improvement of a problem to be solved as perceived by the patient. The program may consist of the commitment on a series of supervised uses of the treatment with evolution of the exposure parameters (frequency, power, etc.). A discovery session, followed by a session adapted to the patient's feeling and the power of the effect perceived could be envisioned. The following sessions could also be adapted based on the measurement of said effect if sensors allow to measure it. Lastly, the treatment session could be triggered by the user through a program, or automatically if sensors allow to measure the need thereof.

Naturally, several modifications may be made to the invention without departing from the scope thereof.

According to aspects, a unit (e.g., the device 1000) is disclosed that emits millimeter waves (MMW) to a patient, which cause the central release of endogenous opioids to decrease pain and induce sleep for the patient. For example, MMWs stimulate subcutaneous nerve receptors of the patient (e.g., at the patient's wrist, or wherever skin contact occurs on the patient), sending a message to the brain, which in turn releases endorphins.

Endorphins are natural opioids and their release varies throughout the day and according to the stimulation received by the peripheral nervous system. Thus, painful (e.g. nociceptive stimuli, pregnancy and childbirth) and non-painful (e.g. temperature, massage, light) stimuli lead to an increase in endorphin levels. Endorphins are involved in pain regulation both by reducing ascending transmission of the nociceptive message and by descending inhibition from the brain to the spinal cord. At the peripheral level, endogenous opioids reduce the ascending transmission of nociceptive impulses. At the central level, endogenous opioids reduce interneural signal transmission of the nociceptive message and inhibit the release of GABA, thus resulting in abundant release of dopamine. Dopamine is a main actor in the feelings of pleasure, reward and euphoria and as such modulates the perception of pain, especially its affective and motivational aspects. Endogenous opioids also influence the balance between sympathetic and parasympathetic systems namely by inhibition of the β-adrenergic activity which results in a modulation of breathing and heartbeat. In addition, endogenous opioids also regulate the cholinergic activity, which is strongly involved in one's level of arousal. At the behavioral level, the release of endorphins triggers sleep onset.

According to certain aspects, MMW emitters may be miniaturized to fit into a wristband (e.g., the device 1000) wearable by patients for an autonomous at-home use. In an implementation, patients may perform three MMW therapy sessions of 30 minutes each, using a therapeutic wristband (e.g., the device 1000) everyday for 3 months in order to improve their quality of life (e.g., less pain and better sleep). Poor sleep and pain are debilitating and strongly impact patients' quality of life. The regular use of MMW for therapy improves the patient's sleep and reduces their pain, thereby improving their quality of life. Other therapy session lengths and overall treatment durations can also be used without departing from the scope of the invention.

As an electronic medication relying on one's own resource, there's no risk of overdose, nor any other side effects. As a wearable device, the therapeutic wristband is an at-home treatment that can be used at the patient's convenience. The patients are thus completely autonomous and responsible for their own treatment in terms of time and frequency, an aspect that is poorly addressed amongst the current solutions offered to fibromyalgia patients.

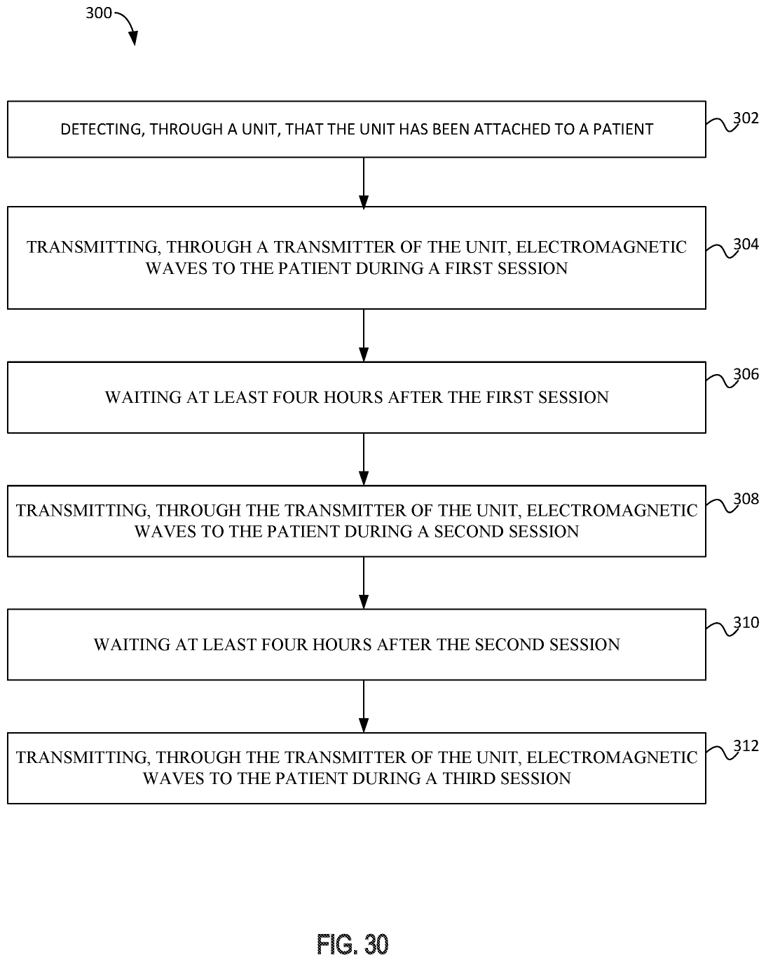

FIG. 30 illustrates a flow chart of an example process 300 for treating fibromyalgia symptoms, according to certain aspects of the disclosure. For explanatory purposes, some blocks of the example process of FIG. 30 are described herein as occurring in series, or linearly. However, multiple blocks of the example process of FIG. 30 may occur in parallel. In addition, the blocks of the example process of FIG. 30 need not be performed in the order shown and/or one or more of the blocks of the example process of FIG. 30 need not be performed.

At block 302, a unit detects that the unit has been attached to a patient. For example, the unit may include the device 1000 of FIGS. 1, 19, and 20. According to an aspect, the device 1000 may be worn by (e.g., attached to) a patient (e.g., a fibromyalgia patient).

At block 304, a transmitter of the unit transmits electromagnetic waves to the patient during a first session. For example, the patient may wear the device 1000 on their wrist, and the device 1000 may transmit millimetric waves (MMW) through MMW emitters. According to aspects, stimulating a wrist of the patient with MMW leads to central release of endogenous opioids, which decrease pain and induce sleep. In an implementation, the unit may have a power flux density of at least 0.5 mW/cm² of skin and a frequency value between 3 and 120 GHz.

At block 306, the patient waits at least four hours after the first session has ended. For example, the first session may have lasted about 30 minutes, though other session lengths may also be employed that are greater than or less than 30 minutes.

At block 308, the transmitter of the unit transmits electromagnetic waves to the patient during a second session. For example, the second session may last about 30 minutes. Other session lengths may also be employed that are greater than or less than 30 minutes, though a shorter session length may also be employed.

At block 310, the patient waits at least four hours after the second session has ended.

At block 312, the transmitter of the unit transmits electromagnetic waves to the patient during a third session. For example, the third session may last 30 minutes. Though other session lengths may also be employed that are greater than or less than 30 minutes.

According to aspects, at least one of the sessions may occur around or at bedtime of the patient. According to aspects, the sessions may occur every day for at least three continuous months, but it could also be longer or shorter than three continuous months.

According to additional aspects, the unit (e.g., the device 1000) may track the patient's daily activities in order to determine when to deliver the treatment (e.g., a session). For example, the unit may track the patient's waking-up time, bedtime, amount of total daily activity, number of steps, etc. The unit may also average the values of the tracked daily activities. In an implementation, the unit may automatically trigger the treatment at the right time, based on how active the patient was during that day. For example, thresholds may be established for the patient based on the patient's average daily activity level. If the patient crosses any of the thresholds, then the unit may be automatically triggered to administer the treatment (e.g., begin a session). The unit may also track how often the treatment has been given in a 24-hour period, in order to prevent over-usage of the treatment. For example, the unit may be prevented from administering a treatment for a specified timeout period (e.g., four hours, or otherwise) such that it may not administer another treatment until expiry of the specified timeout. In this way, the patient may go about their day without having to actively track their own activities.

It is understood that any of the first, second, and/or third sessions may be been longer or shorter than 30 minutes. It is further understood that the patient may wait longer or shorter than 4 hours between the sessions. It is further understood that the patient may undergo more or less than three sessions. According to aspects, a maximum session threshold may be five sessions in a 24-hour period.

The invention also relates to a computer-implemented method for treating fibromyalgia symptoms that includes detecting, through a unit, that the unit has been attached to a patient, transmitting, through a transmitter of the unit, electromagnetic waves to the patient during a first session, waiting at least four hours after the first session, transmitting, through the transmitter of the unit, electromagnetic waves to the patient during a second session, waiting at least four hours after the second session, transmitting, through the transmitter of the unit, electromagnetic waves to the patient during a third session.

The invention also relates to a non-transitory computer-readable medium that stores instructions that, when executed by a processor, cause the processor to perform a method for treating fibromyalgia symptoms that includes detecting, through a unit, that the unit has been attached to a patient, transmitting, through a transmitter of the unit, electromagnetic waves to the patient during a first session, waiting at least four hours after the first session, transmitting, through the transmitter of the unit, electromagnetic waves to the patient during a second session, waiting at least four hours after the second session, transmitting, through the transmitter of the unit, electromagnetic waves to the patient during a third session.

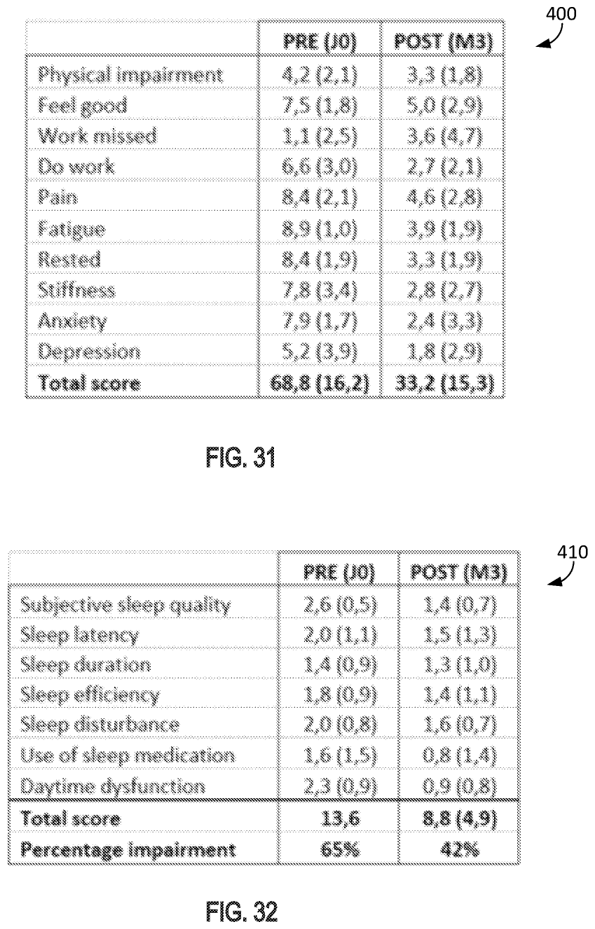

FIGS. 31 and 32 illustrate results of a fibromyalgia treatment program according to the invention. Referring to FIG. 31, a table 400 illustrates the results of a 3-month fibromyalgia treatment program from utilizing the device 1000. As shown, on average, the patients experienced a 52% improvement of their Fibromyalgia Impact Questionnaire (FIQ) scores.

Referring to FIG. 32, a table 410 shows illustrates the results of a 3-month fibromyalgia treatment program from utilizing the device 1000. As shown, on average, the patients experienced a 36% improvement in their Pittsburgh Sleep Quality Index (PSQI) scores.

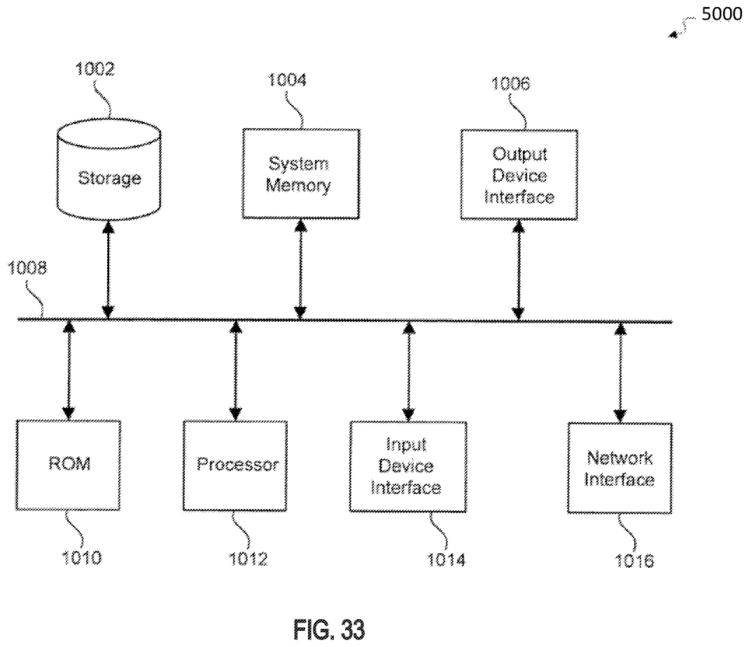

FIG. 33 conceptually illustrates electronic system 5000 with which one or more aspects of the subject technology may be implemented. Electronic system 5000, for example, may be, or may be a part of, a portable electronic device such as a laptop computer, a tablet computer, a phone, a wearable device, a personal digital assistant (PDA), a vehicle, or generally any electronic device that transmits signals over a network. Such an electronic system includes various types of computer-readable media and interfaces for various other types of computer-readable media. Electronic system 5000 includes bus 1008, processing unit(s) 1012, system memory 1004, read-only memory (ROM) 1010, permanent storage device 1002, input device interface 1014, output device interface 1006, and network interface 1016, or subsets and variations thereof.

Bus 1008 collectively represents all system, peripheral, and chipset buses that communicatively connect the numerous internal devices of electronic system 5000. In one or more embodiments, bus 1008 communicatively connects processing unit(s) 1012 with ROM 1010, system memory 1004, and permanent storage device 1002. From these various memory units, processing unit(s) 1012 retrieves instructions to execute and data to process in order to execute the processes of the subject disclosure. The processing unit(s) can be a single processor or a multi-core processor in different embodiments.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system 5000 is off. One or more embodiments of the subject disclosure uses a mass-storage device (such as a magnetic or optical disk and its corresponding disk drive) as permanent storage device 1002.

Other embodiments use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such as random access memory. System memory 1004 stores any of the instructions and data that processing unit(s) 1012 needs at runtime. In one or more embodiments, the processes of the subject disclosure are stored in system memory 1004, permanent storage device 1002, and/or ROM 1010. From these various memory units, processing unit(s) 1012 retrieves instructions to execute and data to process in order to execute the processes of one or more embodiments.

Bus 1008 also connects to input and output device interfaces 1014 and 1006. Input device interface 1014 enables a user to communicate information and select commands to the electronic system. Input devices used with input device interface 1014 include, for example, alphanumeric keyboards, pointing devices (also called “cursor control devices”), cameras or other imaging sensors, or generally any device that can receive input. Output device interface 1006 enables, for example, the display of images generated by electronic system 5000. Output devices used with output device interface 1006 include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more embodiments may include devices that function as both input and output devices, such as a touch screen. In these embodiments, feedback provided to the user can be any form of sensory feedback, such as visual feedback, auditory feedback, or tactile feedback; and input from the user can be received in any form, including acoustic, speech, or tactile input.

Finally, as shown in FIG. 31, bus 1008 also couples electronic system 5000 to a network (not shown) through network interface 1016. In this manner, the computer can be a part of a network of computers (such as a local area network (“LAN”), a wide area network (“WAN”), or an Intranet, or a network of networks, such as the Internet. Any or all components of electronic system 5000 can be used in conjunction with the subject disclosure.

Many of the above-described features and applications may be implemented as software processes that are specified as a set of instructions recorded on a computer-readable storage medium (alternatively referred to as computer-readable media, machine-readable media, or machine-readable storage media). When these instructions are executed by one or more processing unit(s) (e.g., one or more processors, cores of processors, or other processing units), they cause the processing unit(s) to perform the actions indicated in the instructions. Examples of computer-readable media include, but are not limited to, RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In one or more embodiments, the computer-readable media does not include carrier waves and electronic signals passing wirelessly or over wired connections, or any other ephemeral signals. For example, the computer-readable media may be entirely restricted to tangible, physical objects that store information in a form that is readable by a computer. In one or more embodiments, the computer-readable media is non-transitory computer-readable media, computer-readable storage media, or non-transitory computer-readable storage media.

In one or more embodiments, a computer program product (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While the above discussion primarily refers to microprocessor or multi-core processors that execute software, one or more embodiments are performed by one or more integrated circuits, such as ASICs or field programmable gate arrays (FPGAs). In one or more embodiments, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon implementation preferences, it is understood that the specific order or hierarchy of blocks in the processes may be rearranged, or that not all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The present invention also relates to a method for treating fibromyalgia and/or one of its symptoms in a human or animal subject, comprising:
the application of a portable device (10; 100; 1000) for transmitting electromagnetic waves to said human or animal subject, wherein said portable device is capable, when it is affixed at a surface (60), of transmitting waves having a power flux density of at least 0.5 mW/cm² of surface and a frequency value of between 3 and 120 GHz, the device being further capable of simultaneously exposing at least 2.5 cm² of the surface to the waves, and
before, during and/or after said application of a portable device, a coaching step.
   The present invention also relates to a method for treating fibromyalgia and/or one of its symptoms in a human or animal subject, comprising:
a step of transmitting electromagnetic waves towards the subject's skin, thanks to a transmitter worn by said subject, said waves having a power flux density of at least 0.5 mW/cm² of skin and a frequency between 3 and 120 GHz, and
before, during and/or after said step of transmitting electromagnetic waves, a coaching step.
   The coaching step includes at least one of the following components:
   (i) providing therapeutic education to the human or animal subject about the device or transmitter used (such as its principle of action, expected effects and/or technical aspects). Said therapeutic education aims to improve adherence, and reduce apprehension and nocebo effects of the human or animal subject. This is particularly important in fibromyalgia;
   (ii) improving compliance and effectiveness, notably thanks to regular assessments of subject's usability, subject's adherence, subject's health benefits and dispensing personalized advice according to assessments, and, for example regarding food or physical activity;
   (iii) discussing between subjects afflicted by fibromyalgia (i.e. peer support), for example through physical meetings or digital forum discussions; and/or
   (iv) collecting data which are to be used by the health practitioner.
   The coaching step may comprise at least one discussion, by telephone, physical or through a digital platform, between the coach and the human or animal subject. The coach is preferably a trained person, preferably a nurse.
   Preferably, the coaching step comprises at least one discussion before said application of a portable device or said step of transmitting electromagnetic waves, and at least one discussion after said application of a portable device or said step of transmitting electromagnetic waves. The coaching step may be digitally automatically provided.
   Preferably, one coaching step occurs before any treatment with the device or transmitter of the invention; and at least one coaching step occurs after the first steps of treatment.

The subject technology is illustrated, for example, according to various aspects described above. The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

### Example 1: Effectiveness of Wristband on Quality of Life and Sleep Quality of People with Fibromyalgia

### Abstract

Background: Fibromyalgia is a long-term disease which causes widespread pain, stiffness, fatigue, amongst other symptoms. There's no cure for fibromyalgia and treatments aim at reducing pain and improving patients' quality of life. While drugs (antalgics, antidepressants, antiepileptics) entail undesirable side effects, non-drug therapies (massages, thermal cures, acupuncture, cryotherapy) are costly and depend on trained staff and/or specific setups. Millimeter wave (MMW) emitters may be miniaturized and fit into a wristband wearable by patients for an autonomous at-home use. The aim of this study was to test the effectiveness of the therapeutic wristband in improving fibromyalgia patient's quality of life and sleep after 3 months of use.

Method: Twenty-one fibromyalgia patients performed 3 MMW therapy sessions of 30 minutes each, using a therapeutic wristband every day for 3 months. Fibromyalgia Impact Questionnaire (FIQ) and the Pittsburgh Sleep Quality Index (PSQI) were completed at J0 and M3.

Results: Preliminary results of the 8 patients who completed their 3-month trial show an average decrease of 52% of their FIQ score, and 36% of their PSQI score.

Conclusions: The daily use of the therapeutic wristband for 3 months led to an improvement in quality of life far superior to what is considered clinically significant and thus seem to be a promising solution to improve fibromyalgia patients' quality of life and sleep.

### Relevance and Aims of the Study

The main symptoms of fibromyalgia are diffuse pain, sleep disorders, chronic fatigue, and mood disorders (anxiety and depression). Sleep and pain are intimately linked: experimental studies obtained in humans and animals show that there is a relationship between disturbances in the physiology of the sleep-wake cycle and diffuse musculoskeletal pain. A poor sleep quality is often followed by an increase in pain intensity the next day. Conversely, a particularly painful day is often followed by a bad night. In healthy subjects, sleep deprivation causes hyperalgesia changes. Healthy subjects report symptoms similar to those reported by people with fibromyalgia (musculoskeletal pain, fatigue and mood disorders). One study shows that repeated sleep interruptions and not only sleep restriction impacts pain inhibition functions and increases spontaneous sensations of pain. Sleep disruptions are more reliable and stronger predictors of pain than pain is of sleep disruption.

Poor sleep and pain are debilitating and strongly impact patients' quality of life. The regular use of MMW therapy could improve patient's sleep, and reduce their pain, and thereby improve their quality of life. The aim of this study was to test the effectiveness of the therapeutic wristband in improving fibromyalgia patient's quality of life and sleep after 3 months of daily use. In this pilot study, the improvement of the Fibromyalgia Impact Questionnaire (FIQ, Perrot et al., 2003) score between before and after the trial was the primary outcome and, following Bennett et al. (2009), the inventors considered a diminution of 14% as clinically significant. Secondary outcomes included score of the Pittsburgh Sleep Quality Index (PSQI), and subsets from the questionnaires FIQ an PSQI.

### Method

1. Participants

Twenty-one participants (20 women, mean age=50.8 years; SD=10.9) diagnosed with fibromyalgia by a physician (mean age of diagnosis m=9.8 years, SD=8.7) were recruited for this study between March and September 2020. The pharmacological treatment of these participants had been stable for at least 1 month at the time of inclusion in the study and remained unchanged for the duration of the study. Amongst these participants, 17 were French, 2 were American and 1 was German. The participants also continued with their usual non-drug treatments and therapeutic activities.

2. Procedure

Participants were included in the trial through their doctor or physiotherapist. At J0, participants were asked to fill some forms including general information (age, location, year of diagnosis, treatment, associated pathologies, analgesic and aggravating factors), the Fibromyalgia Impact Questionnaire (FIQ) assessing the impact of fibromyalgia on quality of life, and the Pittsburgh Sleep Quality Index (PSQI) assessing sleep quality. The results obtained from these questionnaires constituted the “Before” value. Participants were asked to perform 3 sessions of 30 minutes each per day, including one session before bedtime or at bedtime, every day, for 3 months. They could use their wristband up to 5 times a day, with a 4-hour interval between sessions. At the end of the 3-month trial period, participants filled the FIQ and PSQI (“After” value).

Results

Amongst the 21 patients who started, 8 have completed their 3-month trial. Below are the preliminary results for these 8 patients. At this stage, the results show a 52% improvement of the FIQ score, and 36% in the PSQI.

TABLE 1
Mean FIQ subsets and total score, before and after 3-month trial
(values in brackets represents standard deviation of the mean)

| | PRE (J0) | POST (M 3) |
|---|---|---|
| Physical impairment | 4.2 (2.1) | 3.3 (1.8) |
| Feel good | 7.5 (1.8) | 5.0 (2.9) |
| Work missed | 1.1 (2.5) | 3.6 (4.7) |
| Do work | 6.6 (3.0) | 2.7 (2.1) |
| Pain | 8.4 (2.1) | 4.6 (2.8) |
| Fatigue | 8.9 (1.0) | 3.9 (1.9) |
| Rested | 8.4 (1.9) | 3.3 (1.9) |
| Stiffness | 7.8 (3.4) | 2.8 (2.7) |
| Anxiety | 7.9 (1.7) | 2.4 (3.3) |
| Depression | 5.2 (3.9) | 1.8 (2.9) |
| Total score | 68.8 (16.2) | 33.2 (15.3) |

TABLE 2
Mean PSQI subsets and total score, before and after 3-month trial
(values in brackets represents standard deviation of the mean)

| | PRE (J0) | POST (M 3) |
|---|---|---|
| Subjective sleep quality | 2.6 (0.5) | 1.4 (0.7) |
| Sleep latency | 2.0 (1.1) | 1.5 (1.3) |
| Sleep duration | 1.4 (0.9) | 1.3 (1.0) |
| Sleep efficiency | 1.8 (0.9) | 1.4 (1.1) |
| Sleep disturbance | 2.0 (0.8) | 1.6 (0.7) |
| Use of sleep medication | 1.6 (1.5) | 0.8 (1.4) |
| Daytime dysfunction | 2.3 (0.9) | 0.9 (0.8) |
| Total score | 13.6 | 8.8 (4.9) |
| Percentage impairment | 65% | 42% |

### Example 2: Effectiveness of Wristband on Quality of Life and Sleep Quality of People with Fibromyalgia

Abstract

156 fibromyalgia patients started using the MMW emitting wristband according to the invention and coaching, between February and August 2021, performing 3 sessions a day.

They had the possibility to discontinue after 2 months or to carry on with the solution. Responses to the Fibromyalgia Impact Questionnaire (FIQ) and the Pittsburgh Sleep Quality Index (PSQI) were recorded at J0 (PRE-scores) and, for patients who carried on after 2 months, at M3 (POST-scores), Patient Global Impression of Change was recorded at M3.

Amongst the 156 fibromyalgia patients, 122 (78.2%) continued using it after 2 months, while 34 (21.8%) chose to stop. Among 122 patients who continued, 81 accepted to complete POST-questionnaires and 71.6% showed a clinically significant improvement of their FIQ score (i.e. >14%, Bennett, 2009), with a mean decrease of 28.9% of the FIQ scores, and 51.3% showed an improvement of their sleep quality, with a mean decrease of 27.7% of the PSQI scores. Daily use of the device of the invention for 3 months led to an improvement in quality of life over and above what is considered clinically significant and thus seems to be an effective tool for improving fibromyalgia patients' quality of life and sleep.

### Materials & Methods

The device used is the same as in example 1 (i.e. the MMW emitting wristband according to the invention).

Furthermore, patients also received coaching from a health coach trained for this purpose. Coaching has several objectives: First, coaching intends to provide therapeutic education on the device used (principle of action, expected effects, technical aspects) in order to improve adherence and reduce apprehension and nocebo effects that are particularly important in fibromyalgia. Therapeutic education of the patient in the management of fibromyalgia is a recommendation considered “indispensable” (INSERM 2020, p. 50) so that the patient can be “an actor in maintaining or improving his or her quality of life”. To this end, two phone interviews (D0 and D7) were conducted.

The second objective is to improve compliance and effectiveness. Indeed, a meta-analysis evaluating the effects of ecological interventions (EMI: Ecological Momentary Intervention) based on the use of technological tools (websites, apps) on mental health (stress, anxiety and depression) shows that the results can be up to 62% greater when the intervention involves the punctual assistance of a health professional compared to an intervention without a health professional (Versluis et al., 2016). The authors of the study suggest that this assistance could increase users' motivation and adherence, which in turn could increase the effectiveness of the intervention. To this end, a follow-up interview at D45 was conducted.

Finally, the quality of the therapeutic alliance between a patient and a person responsible for providing therapy influences the effectiveness of a treatment. For example, a clinical study demonstrated that electrotherapy reduced pain intensity in a population with chronic low back pain more than a sham device, but also that the effectiveness of this type of therapy increased when the therapeutic alliance was optimized by a therapist who was communicative, technically proficient, offered thoughtful and personalized responses, and demonstrated warmth and support.

The application used in the invention serves as a tool for this personalized coaching. By connecting the wristband to said application, the data on the use of the band is communicated to the coach, who can in turn guide the patient in the use of their band during the scheduled interview.

1. Participants

156 participants (134 women, mean age=53 years; SD=12.1) diagnosed with fibromyalgia by a physician used the Remedee Solution between February and August 2021.

2. Procedure & Outcomes

The wristband of the invention was introduced to participants as a wellness device that could improve their sleep and reduce their stress. At J0, participants were asked to fill out some forms including the Fibromyalgia Impact Questionnaire (FIQ) assessing the impact of fibromyalgia on QoL, and the Pittsburgh Sleep Quality Index (PSQI) assessing sleep quality. The results obtained from these questionnaires constituted the “PRE” scores. Participants were given an hour-long introduction to the scientific principle of MMW therapy, endorphins, the relationship between sleep and pain, and the technical aspects of the device by a health coach (i.e. a person specifically trained to dispense information relative to the device, interact with and support the participants to use their wristband). Participants were asked to perform 3 sessions of 30 minutes each per day, including one session before bedtime or at bedtime, every day. They could use their wristband up to 5 times a day, with a 3-to-4-hour interval between sessions. The best moments to use the wristband during the day were identified by patients and coaches depending on the main symptoms of the patients. At Day 7, they had a phone interview intending to check if the technical aspects were all understood and confirming that the posology agreed was appropriate. At Day 45, during a third phone interview, the coaches qualitatively assessed with the patients if they perceived any benefits. After 2 months, patients were asked if they wanted to keep using their wristband or to stop. Finally, after 3 months of using the Remedee Solution, participants who kept using it were asked to fill the FIQ and PSQI (“POST” scores) and to answer the Patient Global Impression of Change (PGIC, Hurst & Bolton, 2004).

Results

Amongst the 156 patients who were submitted to the method of the invention between February and July 2021, 122 (78,2%) decided to keep using the wristband after 2 months and 34 (21,8%) dropped out. Amongst the 122 who kept using the wristband, 81 accepted to complete the POST-questionnaires, the others declined the request.

I. Quality of Life (FIQ)

Amongst the 81 participants who chose to keep using the solution after 2 months and completed the questionnaires after 3 months, the improvement of mean FIQ scores was 28.9%, from a mean PRE-score of 65.9 (SD=12.9) to a mean POST-score of 46.9 (SD=17.3) (see Table 1). While at the beginning of the trial most patients (61/81 who completed the 3 months) had a FIQ score falling into the “Severe” category, the majority of patients (37/81) got a score falling in the “Mild” category at the end of the trial (Table 2).

TABLE 1
Mean FIQ subsets and total scores, before (PRE) and
after (POST) using the Remedee solution (values in
brackets represent standard deviations of the means)

| | PRE | POST |
|---|---|---|
| Physical Impact | 4.4 (1.9) | 3.2 (1.8) |
| Wellbeing | 8.2 (2.0) | 5.2 (2.8) |
| Work missed | 2.0 (3.3) | 1.7 (3.5) |
| Do work | 6.7 (2.2) | 5.1 (2.4) |
| Pain | 7.6 (1.6) | 5.6 (2.4) |
| Fatigue | 8.1 (1.6) | 5.9 (2.5) |
| Rest | 7.6 (1.9) | 5.3 (2.7) |
| Stiffness | 7.6 (1.9) | 5.4 (2.7) |
| Anxiety | 6.7 (2.5) | 4.6 (3.1) |
| Depression | 4.6 (3.0) | 3.2 (2.9) |
| Total Score | 65.9 (12.9) | 46.9 (17.3) |
| Mean FIQ Improvement | 28.9% | |
| Percentage | | |

TABLE 2
Distribution across severity categories depending on FIQ
score (Mild: scores from 0 to <39; Moderate: scores ≥39
to <59; Severe: scores ≥59; Bennett et al., 2009)

| | PRE | POST |
|---|---|---|
| SEVERE | 75.3% | 21.0% |
| MODERATE | 22.2% | 45.7% |
| MILD | 2.5% | 33.3% |

Following Bennett (2009), the inventors considered that an improvement equal or superior to 14% was clinically significant. Fifty-eight participants (71.6%) showed an improvement superior to 14%, 19 (23.5%) did not show any difference and 4 (4.9%) worsened (i.e. FIQ improvement <−14%). The inventors also ran a one-way repeated measured ANOVA and found a statistically significant decrease of the FIQ scores between before and after the 3 months using the Remedee Solution (F(1,80)=108.7, p<0.001).

Table 1 displays the mean scores across the 81 patients for each of the FIQ subsets before (Pre) and after (Post) the 3 months using the Remedee Solution. Amongst the 81 participants, 35 did not have a professional activity (invalidity or retired), and 46 were workers.

2. Sleep (PSOI)

Amongst the 81 patients who accepted to complete the POST-questionnaires, one forgot to complete the PSQI part, therefore N=80 in Sleep quality analyses.

Before they started using the Remedee solution, 100% of the 80 participants had poor sleep quality, i.e. a PSQI Pre-score >5, as defined by Buysse et al. (1989). After 3 months using the Remedee Solution, the mean PSQI improvement was 27.7% from a mean PRE-score of 13.7/21 (SD=3.7) to a Post-score of 9.9/21 (SD=4.2) (see Table 3).

TABLE 3
Mean PSQI subsets and total scores, before and after
using the Remedee solution for 3 months (values in
brackets represent standard deviations of the means)

| | PRE | POST |
|---|---|---|
| Subjective sleep quality | 2.4 (0.7) | 1.5 (0.7) |
| Sleep latency | 1.9 (1.0) | 1.4 (1.0) |
| Sleep duration | 1.5 (1.0) | 1.1 (0.9) |
| Sleep efficiency | 1.8 (1.2) | 1.3 (1.1) |
| Sleep disturbance | 2.8 (0.4) | 2.5 (0.7) |
| Use of sleep medication | 1.7 (1.4) | 1.1 (1.3) |
| Daytime dysfunction | 1.4 (1.1) | 1.0 (1.1) |
| Total sleep quality score | 13.7 (3.7) | 9.9 (4.2) |
| Percentage sleep impairment | 65% | 47% |
| MEAN SLEEP IMPROVEMENT | 27.7% | |
| PERCENTAGE | | |

Following Jacobson and Truax (1991), the inventors computed the Reliable Change Index (RCI) for each participant to determine if they exhibited a statistically reliable response to treatment. The RCI equation is: RCI=(Post−Pre)/√(2[SDpre√(1−rₓₓ)]²), with: “Pre”=PSQI score before trial; Post”=PSQI score after trial; SD=the group standard deviation of the mean from the pre-trial data; rₓₓ=PSQI test-retest reliability=0.85. Participants with a RCI <−1.96, which would occur by chance less than 5% of the time, were considered to be reliably improved from baseline (PRE) to post 3-month period (POST). In the group of 80 participants who completed the 3-month questionnaires, the denominator of the RCI equation was d=2.0. Forty-one of the 80 participants (51.3%) were reliably improved from Pre to Post trial, and 15 (18.8%) fell into the healthy sleep quality range, with a PSQI score <6. The subjective sleep quality was the most improved questionnaire subset (39.4% improvement), followed by sleep onset latency and daytime functioning.

3. Patient Global Impression of Change

After 3 months of using the Solution, participants were also asked to answer the Patient Global Impression of Change (PGIC, Hurst & Bolton, 2004). The question asked was “Since you started to use the Remedee wristband, how would you describe your health overall?” Table 4 shows the distribution of patients amongst PGIC modalities. Amongst the 81 patients who complemented the POST-questionnaires, 85.2% considered that their health improved, from minimally to very much, 11.1% considered their health as unchanged and 3.7% considered their health as being worse, from minimally to very much, than before they started using the Remedee Solution. Pearson's correlation between the FIQ improvement percentage and PGIC numerical value was statistically significant (r²=0.20; p<0.001) but the correlation between the PSQI improvement percentage and PGIC numerical value was not (r²=0.01; p=0.15).

Table 4
Patient Global Impression of Change. Distribution
of patients amongst verbal categories

| PGIC answers | Percentage patients |
|---|---|
| Very much worse | 1.2% |
| Much worse | 0% |
| Worse | 2.5% |
| No change | 11.1% |
| Minimally improved | 50.6% |
| Much improved | 30.9% |
| Very much | 3.7% |
| improved | |

### CONCLUSION

The method of the invention used in this trial is completely in line with the EULAR's recommendations, in that it is a non-pharmaceutical treatment, that patients are able to use autonomously, having been educated about the mechanisms of MMW therapy and endorphin release, and benefiting from the portable and user-friendly qualities of the wristband. Pairing the use of the wristband with a digital solution providing sleep management programs or adapted physical activity training would be an innovative care way for patients with fibromyalgia in order to promote a change in their global quality of life.

1. A method for treating fibromyalgia and/or at least one symptom of fibromyalgia in a human or animal subject, comprising applying a portable device for transmitting electromagnetic waves to said human or animal subject, wherein said portable device is capable, when it is affixed at a surface, of transmitting electromagnetic waves having a power flux density of at least 0.5 mW/cm² of surface and a frequency value of between 3 and 120 GHz, the device being further capable of simultaneously exposing at least 2.5 cm² of the surface to the electromagnetic waves.

2. A method for treating fibromyalgia and/or one of its symptoms in a human or animal subject, which comprises a step of transmitting electromagnetic waves towards the subject's skin, wherein the electromagnetic waves are transmitted from a transmitter worn by said subject, said electromagnetic waves having a power flux density of at least 0.5 mW/cm² of skin and a frequency between 3 and 120 GHz.

3. The method according to claim 1, wherein the electromagnetic waves have a power flux density of between 5 and 35 mW/cm².

4. The method according to claim 1, wherein the portable device comprises a unit that detects human or animal skin, the portable device being able to signal the presence or absence of skin to be exposed to electromagnetic waves, and/or to determine a distance separating the skin and the portable device.

5. The method according to claim 1, wherein the portable device is suitable for being worn at least in one of the following places: around a wrist;
on one leg;
on an ankle;
on a back;
on an ear; or
in the palm of a hand.

6. The method according to claim 1, wherein said at least one symptom of fibromyalgia is chosen from (i) pain, (ii) stiffness, (iii) tenderness of muscles, tendons, and joints in the absence of inflammation, (iv) sleep impairments, and (v) fatigue/lack of rest.

7. The method according to claim 3, wherein the electromagnetic waves have a power flux density of between 5 and 15 mW/cm².

8. The method according to claim 1, wherein the electromagnetic waves have a frequency between 60 and 95 GHz, or between 61 and 61.5 GHz.

9. The method according to claim 1, wherein the portable device is a wristband.

10. The method according to claim 1, wherein the subject is human.

11. The method according to claim 1, wherein the surface is the subject's skin.

12. The method according to claim 2, further comprising
detecting the subject's skin prior to the step of transmitting.

13. The method according to claim 1, which comprises, before, during and/or after applying the portable device, at least one step of coaching the subject with respect to using the portable device.

14. The method according to claim 2, which comprises, before, during and/or after transmitting the electromagnetic waves, at least one step of coaching the subject with respect to using the portable device.

15. The method according to claim 13, wherein the at least one coaching step comprises
(i) providing therapeutic education to the human or animal subject about the portable device or transmitter that is used; and/or
(ii) improving adherence and effectiveness, by conducting regular assessments of the subject's ease of using the portable device, the subject's adherence to use of the portable device, and the subject's health benefits from using the portable device, and then dispensing personalized advice according to the regular assessments; and/or
(iii) discussing use of the portable device among subjects afflicted by fibromyalgia; and/or
(iv) collecting data to be used by a health practitioner.

16. The method according to claim 14, wherein the at least one coaching step comprises conducting at least one discussion, by telephone, in person or through a digital platform, between a coach and the human or animal subject.

17. The method according to claim 16, wherein the at least one coaching step comprises conducting at least one discussion before applying the portable device or transmitting the electromagnetic waves, and conducting at least one discussion after applying the portable device or transmitting the electromagnetic waves.

18. The method according to claim 17, wherein one coaching step occurs before applying the portable device or transmitting the electromagnetic waves; and at least one additional coaching step occurs after applying the portable device or transmitting the electromagnetic waves.
